(19) United States
(12) Patent Application Publication
(10) Pub. No.: US 2024/0044893 A1
(43) Pub. Date: Feb. 8, 2024

(54) ULTRASENSITIVE SENSING METHOD FOR DETECTION OF BIOMOLECULES

(71) Applicant: University of Connecticut, Farmington, CT (US)

(72) Inventors: Yu Lei, Storrs, CT (US); Haomin Liu, Farmington, CT (US); Yikun Huang, Farmington, CT (US)

(21) Appl. No.: 18/351,317

(22) Filed: Jul. 12, 2023

Related U.S. Application Data
(60) Provisional application No. 63/388,354, filed on Jul. 12, 2022.

(51) Int. Cl. G01N 33/569; G01N 33/553; G01N 33/543

(52) U.S. Cl. CPC G01N 33/56916; G01N 33/553; G01N 33/54306; G01N 2333/245; G01N 2469/10

(57) ABSTRACT

A method for detecting a target molecule in a sample includes providing a capture species immobilized on a fixed surface; exposing the immobilized capture species to the sample to bind the target molecule; exposing the bound target molecule to a reporter complex to bind the reporter complex to the bound target molecule; and imaging the fixed surface to count the number of the reporter complexes or the number of an imageable product derived from each reporter complex. In an aspect, the signal of the bound target molecule can be amplified to provide an amplified signal that is then exposed to the reporter complex.

### CROSS-REFERENCE TO RELATED APPLICATION

This application is a nonprovisional filing of and claims priority to U.S. Patent Application No. 63,388, filed Jul. 12, 2022, the entire content of which is incorporated herein by reference.

### BACKGROUND

### 1. Field of the Invention

This application relates to ultrasensitive immunoassay-based detection methods for biomolecules.

### 2. Description of the Related Art

Immunoassay has been a dominant technique in biomarker detection since its emergence in the 1960s. Specifically, enzyme-linked immunosorbent assay (ELISA), developed in 1971, rapidly became the gold standard for detecting biomolecules. Because of its convenience and wide applications in various fields, the improvement of ELISA performance started almost at the same time that it was developed, including, for example, fluorescence-based ELISA, and beads-based ELISA to detect antigens. In the past two decades, a variety of different ELISA techniques have been further reported, including digital ELISA for biomarker detection at a single molecule level, paper-based ELISA for fast diagnosis and low-cost point of care, and nano-ELISA for food analysis. ELISA has been used for the detection of emerging targets such as SARS-CoV-2.

Although these techniques are useful, the dominant ELISA format used in clinical practice remains conventional plate-based ELISA because of its low instrument requirements, high reliability, affordability, adaptability, and convenience. However, conventional plate-based ELISAs typically detect the concentration of biomarkers at the level of 10⁻² M and above, and thus may not be able to offer the very low limit of detection (LOD) desired for early diagnosis of disease-related biomarkers (e.g., HIV and cancers). It is challenging to improve the performance of conventional plate-based ELISA with minimal modifications of the protocols used, and that can be readily adapted to the clinical practice using current commercially available ELISA kits. One approach has been to introduce nanoparticles (e.g., metal, silicon, biological nanoparticles) into current conventional plate-based ELISA for signal enhancement. However, disadvantages of this strategy, such as the requirement for nanoparticles and the variation of nanoparticles' quality from batch to batch may result in low reproducibility, thus decreasing their use in a wide variety of applications. Another approach has been to develop new substrate molecules for enhanced signals. However, application to plate-based ELISA requires that the substrate molecules be easily accessible, and the synthesis of the substrate is an extra cost. Accordingly, there remains a need in the art for plate-based ELISA techniques with improved sensitivity.

### SUMMARY

A method for detecting a target molecule in a sample includes providing a capture species immobilized on a fixed surface; exposing the immobilized capture species to the sample to bind the target molecule; exposing the bound target molecule to a reporter complex to bind the reporter complex to the bound target molecule; and imaging the fixed surface to count the number of the reporter complexes or the number of an imageable product derived from each reporter complex. In an aspect, the signal of the bound target molecule can be amplified to provide an amplified signal that is then exposed to the reporter complex.

The above described and other features are exemplified by the following figures, detailed description, examples, and claims.

### BRIEF DESCRIPTION OF THE DRAWINGS

The following FIGURES are exemplary embodiments, which are provided to illustrate the present disclosure. The FIGURES are not intended to limit and are not intended to limit devices, processes, uses, materials, conditions, or process parameters set forth herein.

FIG. 1 is a schematic diagram of an embodiment of signal amplification as described herein.

FIG. 2 is a schematic diagram of an embodiment of the detection method using signal amplification as described herein, in particular the detection of single binding event using an enzymatic reaction to generate a water-insoluble reporter.

FIG. 3A, FIG. 3B, and FIG. 3C are schematic diagrams showing three exemplary embodiments of capturing a target biomolecule and subsequent biosensing via fluorescent dot counting.

FIG. 4 is a schematic illustration of an aspect of detection using a reporter complex including a bacterium with a surface-expressed antibody-binding domain as a universal signal amplifier antigen.

FIG. 5 shows the reaction of ELFP to ELFA.

FIG. 6 (top panel) is a schematic diagram showing detection of a target biomolecule with single molecule sensitivity using an exemplary embodiment of a biotin-streptavidin-alkaline phosphatase reporter complex, and FIG. 6 (bottom panel) shows photographs of scanning microscope images showing single binding events of the slide (left), a zoom-in ratio of 25% (middle) and a single view (right).

FIG. 7 (top panel) is a schematic diagram showing a magnetic bead attached to a capture antibody and a reporter complex including alkaline phosphatase directly attached to an antibody. FIG. 7 (bottom panel) is a schematic diagram showing a magnetic bead attached to a capture antibody, in combination a biotin-streptavidin-alkaline phosphatase reporter complex.

FIG. 8 (top panel) is a schematic diagram showing sandwich immunodetection of a target biomolecule with single molecule sensitivity using fluorescent beads, and FIG. 8 (bottom panel) is photograph of scanning microscope images of the reporter complex product of the slide (left), a zoom-in ratio of 25% (middle) and a single view (right).

FIG. 9 shows a scanning large image of the sensing area for PSA detection, showing a fluorescent image (left panel) and a transmitted image (right panel).

FIG. 10 (top panel) is a schematic diagram showing sandwich immunodetection of a target biomolecule with single molecule sensitivity using magnetic and fluorescent beads conjugated to the capture and detection antibodies, respectively, and FIG. 8 (bottom panel) are photographs of scanning microscope images of the reporter complex product of the slide (left), a zoom-in ratio of 25% (middle) and a single view (right).

FIG. 11 (top panel) shows a schematic diagram illustrating use of a bacterium with a surface-expressed antibody-binding domain as the reporter complex, and FIG. 11 (bottom panel) are photographs of scanning microscope images of the immunocomplexes-reporter bacteria of the large image (left), at a zoom-in ratio of 25% (middle), and a single view (right).

FIG. 12A is a photograph of a bright field image and FIG. 12B of a fluorescence image of antibody captured Z-domain expressed E. coli before staining, and FIG. 12C is a bright field image and FIG. 12D a fluorescent image after Nile red staining.

FIG. 13 is a schematic showing the TSA reaction.

FIG. 14 is a schematic diagram of an embodiment of a dual-amplification counting (DAC) ELISA strategy.

FIG. 15 shows photographs of scanning microscope images showing a comparison of the effect of ELFP substrate and ELFA-saturated ELFP substrate on fluorescent precipitates formation, in particular representative field-of-view (FOV) images of a well incubated with 16.67 μM ELFP substrate at (A) 1.5 hours, (B) 2 hours, (C) 2.5 hours, and (D) 3 hours, respectively; and representative FOV images of a well incubated with 16.67 μM ELFP substrate (ELFA saturated) at (E) 1.5 hours, (F) 2 hours, (G) 2.5 hours, and (H) 3 hours, respectively. The analyte was IgG standard with a concentration of 1.5625 ng/mL, and the dual amplification protocol was applied.

FIG. 16 (top panel) are graphs showing the performance of an embodiment of the DAC ELISA method for mouse IgG detection using (A) HRP-TMB reporter system, (B) HRP-TSA-TMB reporter system, and (C) DAC reporter system; and (middle and bottom panels) one representative image at each IgG concentration, in particular (D) 0 (negative control), (E) 0.0977 ng/mL, (F) 0.1953 ng/mL, (G) 0.3906 ng/mL, (H) 0.7813 ng/mL, (I) 1.5625 ng/mL—used to establish the calibration curve of DAC-ELISA system.

FIG. 17A and FIG. 17B are graphs showing the matrix effect and recovery tests of DAC-ELISA, where FIG. 17A shows background signal from mouse-IgG free goat serum at varied dilutions, and FIG. 17B shows recovery tests using mouse IgG spiked into 100,000× diluted goat serum with the final concentrations of 0 ng/mL (control), 0.2 ng/mL, and 0.1 ng/mL (n=3).

FIG. 18 shows photographs of scanning results for determination of the substrate incubation time, showing one representative image for different incubation times at (A) 1 hr, (B) 2 hr, (C) 3 hr, and (D) 4 hr, respectively. The analyte was IgG standard with a concentration of 1.5625 ng/mL. The substrate was 16.67 μM ELFP solution (ELFA saturated), and the dual amplification method was applied.

FIG. 19 shows photographs f scanning microscope images of fluorescent precipitate distribution in a typical well. (A) One image of a whole well after DAC-ELISA. One representative image corresponds to the spots in the whole well image: (B) spot 1, (C) spot 2, (D) spot 3, (E) spot 4, and (F) spot 5. The analyte was IgG standard with a concentration of 1.5625 ng/mL and the dual amplification method was applied.

### DETAILED DESCRIPTION

Disclosed herein is an ultrasensitive biomolecule detection method that uses plate-based ELISA technology in combination with intrinsically simple, but powerful, signal amplification and detection strategies. In particular, the biomolecule detection method includes efficient, plate-based ELISA technology in combination with unique signal amplifiers (e.g., enzyme-enabled fluorescent dot generation; fluorescent nano/microbeads; antibody-binding domain functionalized E. coli) and area-imaging technology (e.g., scanning microscopy) for quantitation. In an aspect, the method uses dual amplification, in particular biotinyl-tyramide (B-T) conjugation to increase the number of biotins, which further increases the number of reporter enzymes that conjugate to biotin.

The method allows detection of a broad range of biomolecules, including peptides, proteins, enzymes, DNA, RNA, viruses, and bacteria, for example pathogenic bacteria. The dynamic range of the method can be broad, and an ultra-low number of biomarkers can be detected. The detection of single binding event has been achieved, thus allowing ultrasensitive detection of single biomolecule. The detection of about 10³ molecules per microliter (equivalent to 1.6 femtomole (fM)) indicates that the imperceptible concentration of biomarkers associated with some diseases could be easily detected in an early stage using this method. The effectiveness and efficiency of the method suggests that there is significant potential to commercialize it for a wide variety of applications, particularly in diagnosis and detection.

A further advantage is that the detection method is compatible with most, if not all, existing ELISA testing procedures. For example, it can be compatible with current immunoassays and/or DNA assays already existed in the market. The materials for the method are widely available, and have a low cost. The total detection time of can be from 1 to 3 hours at single biomolecule sensitivity level, depending on the type of signal amplifier used. It is also possible to achieve a batch of assays or multiplexed assays for a large number of samples.

The detection method relies on first, signal amplification using a reporter complex that provides an imageable result; followed by large-area imaging and counting to detect the amplified signal. In an aspect, the signal amplifiers can be enzyme-enabled fluorescent dot generation; fluorescent nano/microbeads; antibody-binding domain functionalized E. coli) that are analyzed by large area-imaging technology (e.g., scanning microscopy) for quantitation.

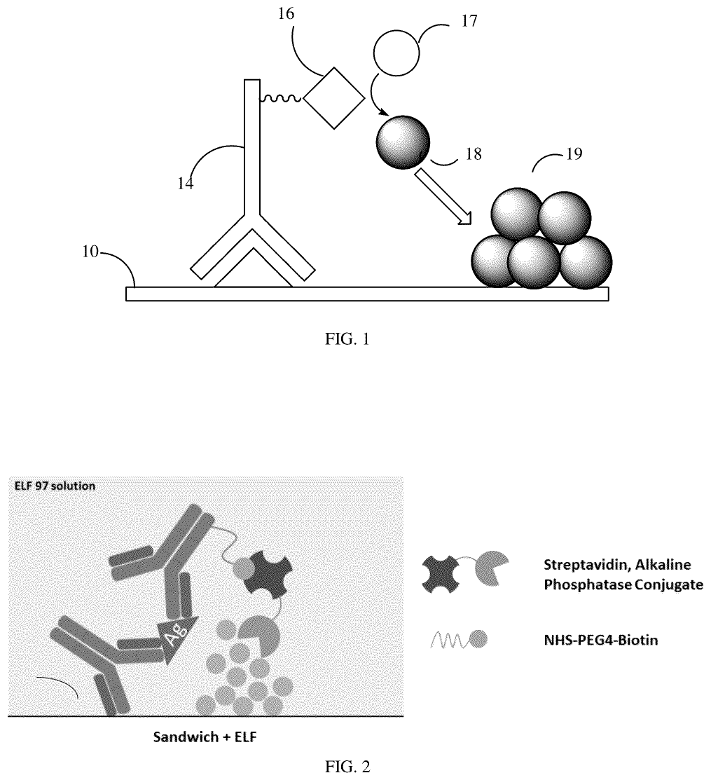

Conventional methods for signal amplification are based on enzyme reactions to generate water-soluble reporters (e.g., fluorophores or chemicals having a detectable color) that will diffuse to the entire solution, which results in significant dilution of the reporter. In contrast, in an embodiment, the current method uses an enzymatic reaction to generate a water-insoluble reporter. As illustrated in an embodiment in FIG. 1, a target macromolecule (here, an antigen 12 from a sample to be analyzed) is immobilized on a solid, fixed surface 10 of, e.g., a microplate well. As used herein, a “fixed surface” means a surface that can be readily imaged, such as a glass slide, microwell of a microplate, or a microplate. The “fixed surface” may include, for example, microbeads or nanobeads, provided that they are immobilized. The antigen 12 is then exposed to a reporter conjugate, here an antibody 14 that complexes with the antigen, and which is itself linked to a reporter enzyme 16. Detection is accomplished by measuring the activity of the reporter enzyme in providing a report molecule, for example via incubation with a fluorogenic substrate 17 that provides a fluorescent, water-insoluble product 18 that is detectable by microscopy. In an aspect, as shown in FIG. 1, the water-insoluble reporters deposit locally from an aqueous solution and aggregate to a larger particle 19 that can be visualized using, for example, a scanning microscope. A scanning microscope can be used to count the number of fluorescent dots in the large area to directly obtain the number of biomolecules without any additional signal amplification. For example, a scanning microscope under 20× or 40× magnification can be used to collect the images over large test area to count the number of reporters, which can be correlated to the number and concentration of target molecules. In another aspect, imaging chips can be used instead of a scanning microscope to achieve similar results. For example, snapshot-based on-chip imaging techniques can be used that can image a large area or even the entire microplate wells in a short time. In still another aspect, a representative portion of the fixed surface can be imaged.

In this method, one such visual particle can correspond to one immunobinding event (and thus one captured target molecule), thus achieving ultra-sensitive detection of targets with single molecule sensitivity. Use of the image-based direct-counting methods described herein substantially avoids resolution issues of the prior art, in that the sensitivity of a fluorescent microscope is so high that individual ELFA precipitates at the well bottom are detectable and contribute to a higher assay sensitivity. The lower limit of detection using this approach can be, for example, 0.01 femtomole (fM), or 0.1 fM, or 1.0 fM.

This amplification and counting method can be applied to a plate based technique as illustrated in FIG. 2. FIG. 2 shows an antibody 21 immobilized on a fixed surface 22 of, e.g, a microplate well. The antibody 21 is then exposed to a target macromolecule (here, an antigen 20 from a sample to be analyzed), which complexes with antibody 21. The complex is exposed to a reporter complex, here a detection antibody 23 conjugated to biotin 24 via an N-hydroxysuccinimide (NHS)-polyethylene (PEG4) linker 25. The biotin 24 is itself conjugated to a streptavidin-alkaline phosphatase conjugate 25. Detection is accomplished by measuring the activity of the reporter enzyme (the alkaline phosphatase) in providing a reporter molecule, for example via incubation with a fluorogenic substrate that provides a fluorescent, preferably water-insoluble product 26 that is detectable by microscopy. In an aspect, the water-insoluble reporters 26 deposit locally from an aqueous solution and aggregate to a larger particle that can be visualized using, for example, a scanning microscope. Alternatively, one such visual particle can correspond to one immunobinding event (and thus one captured target molecule), thus achieving ultra-sensitive detection of targets with single molecule sensitivity.

The ultrasensitive detection method is not limited to enzymatic reaction or fluorescence change. For example, a precipitate with a detectable color generated through enzymatic reaction could also be used, for example, horse radish peroxidase and 3,3′-diaminobenzidine (DAB)). Chemical reaction can also be applied to generate such large particles reporter for similar detection purpose.

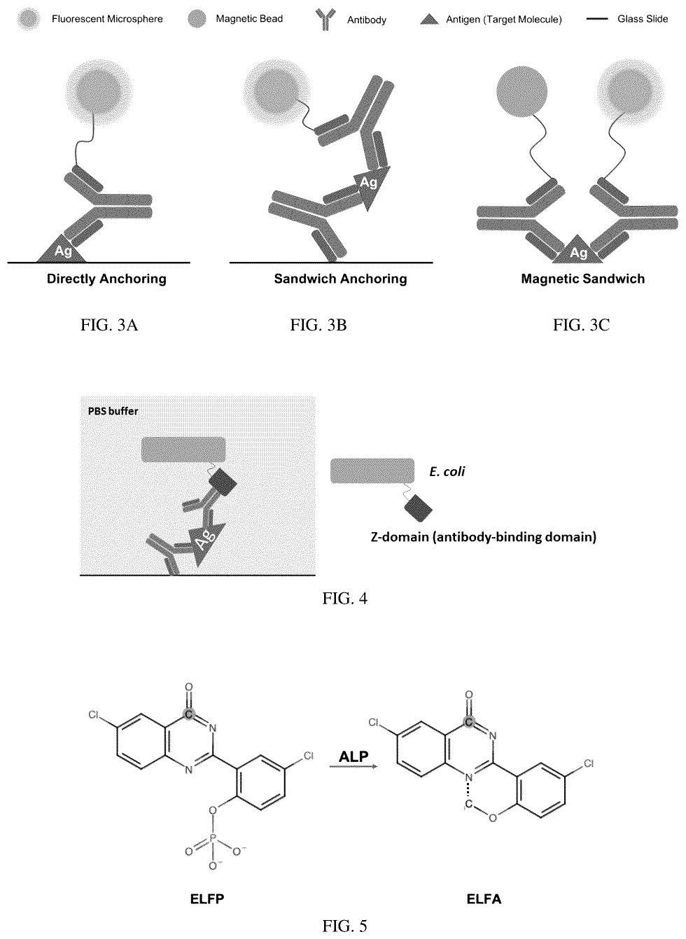

FIG. 3 illustrates other amplification and detection methods. As shown in FIG. 3A and 3B (and referencing the method of FIG. 1 and FIG. 2, respectively), an antibody 30 is directly linked to a fluorescent or colored reporter, for example a fluorescent microbead or nanobead 32. As is understood in the art, a “bead” can have any shape, for example spherical, oval, or irregular. As shown in FIG. 3C, a second antibody 34 that binds to the antigen is linked to a second reporter, for example a magnetic microbead or nanobead 36. In this strategy, detecting antibody labelled with fluorescent reporter (nano- or microbeads) into a capture-antibody functionalized glass chamber is performed without separation and isolation of the biomolecules, which can be tedious and time-consuming. As described above, a scanning microscope can be used to image and digitally count the number of fluorescent dots with single biomolecule sensitivity in the large area to direct obtain the number of biomolecules without any additional signal amplification. The counting-based signal detection strategy can overcome the intrinsic resolution limitation of the instrumentation, which is the major barrier in conventional (e.g., colorimetric) plate-based ELISA. The reporter can be fluorescent beads as well as gold nanoparticles (after gold enhancement reaction). Use of a magnetic bead as shown in FIG. 3C with a capture antibody can enhance capture and separation efficiency.

In another aspect of the ultrasensitive detection method, the reporter complex is a bacterium with a surface-expressed antibody binding domain, because the size of single bacterium (e.g., E. coli) is large enough to be observed using a conventional scanning microscope. In this signal amplification technique, as illustrated in FIG. 4, bacterium such as coli with a surface-expressed antibody-binding domain (Z domain) is used as the reporter complex. After immunoreaction and the binding of E. coli through the Z-domain, the number of bacteria can be directly counted using scanning microscope and correlated to the concentration of target molecules. The bacteria can also either be stained to have fluorescence (using, e.g., Nile red) or be modified to include self-expressing reporters (e.g., fluorescent proteins) for better resolution. Such bacterial reporter complexes can be readily prepared through cell culturing with low cost.

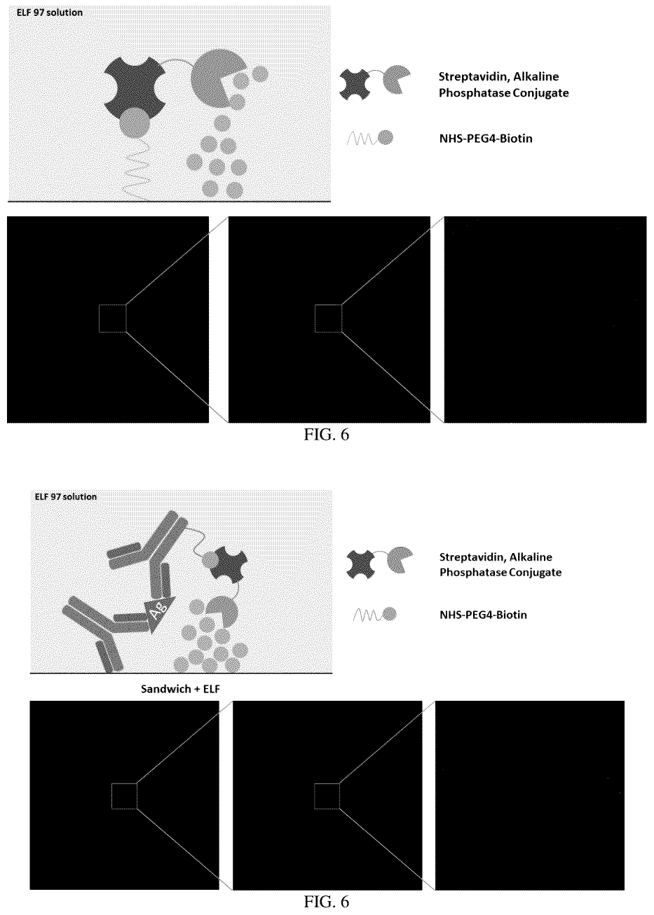

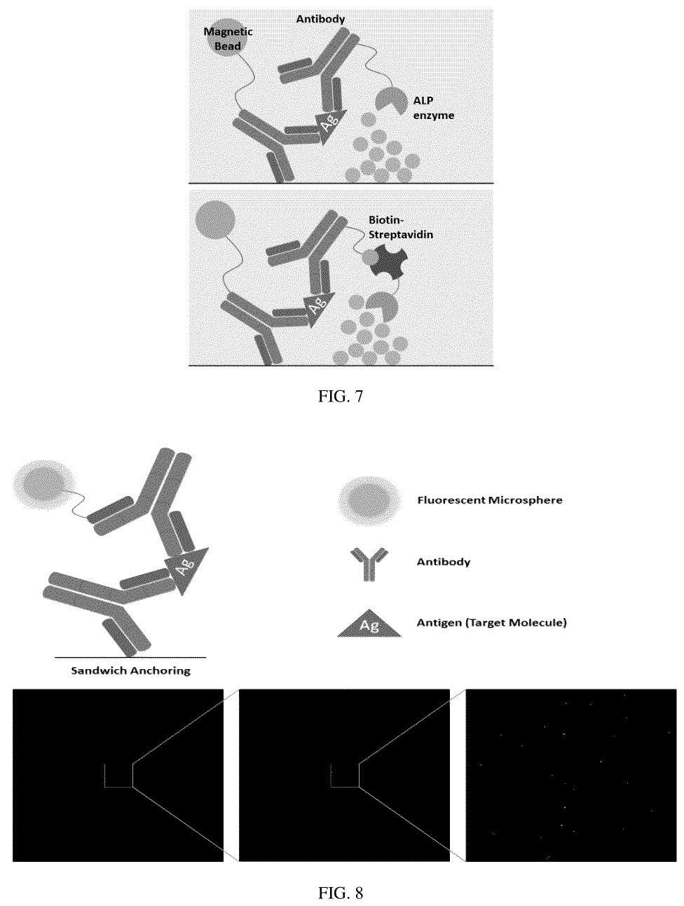

This method has been demonstrated using a reporter complex including a biotin-alkaline phosphatase (ALP) reporter complex and the substrate 2-(5′-chloro-2-phosphoryloxyphenyl)-6-chloro-4(3H)-quinazolinone, also known as ELFP or ELF 97. The dephosphorylated product of this enzyme reaction is known as ELF alcohol (ELFA), as shown in FIG. 5. In this example, a glass-bottomed 96-microplate is used as the reaction chamber. The chamber is rinsed with ethanol and KOH solution successively to remove any dust on the glass surface and enhance its hydrophilicity. After drying the glass surface by nitrogen blowing, it is treated with oxygen plasma to introduce hydroxy groups for reaction with (3-aminopropyl)triethoxy silane (APTES), which provide amino groups to the surface. The amino-group treated surface was exposed to biotin linked to N-hydroxysuccinimide via a 4-unit polyethylene glycol (NHS-PEG4-Biotin (aq)) to functionalize the bottom surface with biotin. After biotin functionalization (biotinylation) is completed, a streptavidin-alkaline phosphatase conjugate is introduced (FIG. 6) to form the streptavidin-biotin complex, thus anchoring the alkaline phosphatase on the surface. Then, the addition of the substrate for alkaline phosphatase (ELF-97) generated water-insoluble fluorescent molecules through enzymatic hydrolysis results in bright and photostable yellow-green fluorescent aggregates (preferably as precipitates) at the site of each enzyme. Once the aggregate grows to a specific size, it can be imaged via an optical microscope, as shown in FIG. 6. Each fluorescent dot is attributed to one single enzymatic activity, and thus accounts for one binding event to the biotin.

In another aspect, it is possible to limit diffusion/movement of the aggregate by gelling the immunocomplex. For example 30 mg gelatin is first dissolved in one milliliter (mL) of deionized (DI) water in a 37° C. water bath until the powder is fully dissolved. Then 100 microliter (μL) of 30 milligrams (mg)/mL gelatin water solution is added to cover the surface with immunocomplexes, to form a transparent gel layer, generally within 20 minutes. After the gel is formed, 1000 μL ELF 97 solution is loaded on the top of the gel to develop the fluorescent dots through enzymatic reaction. In this way, the enzyme is completely immobilized by the gel and will not move, and neither doe the aggregates.

The alkaline phosphatase-ELF 97 reaction (or other reporter complex and substrate) can be used to characterize a single immunocomplex formed within conventional ELISA, which allows counting, in particular digital counting of the actual number of target molecules captured in, for example, a slide or a microplate well. For example, an ELISA can be conducted in accordance with an ordinary protocol, with the addition of signal amplification step. An embodiment is shown in FIG. 6, using immunoglobulin G ((IgG), prostate specific antigen (PSA), and IL-2 as model ELISA assays, and the above-described biotin-alkaline phosphatase as a reporter system. PSA is widely used in the diagnosis of prostate cancer.

Accordingly, an IL-2 capture antibody is first anchored on the surface of 96 microplate. After the antibody anchoring, blocking buffer (BSA solution) is loaded to block the uncovered area and eliminate any nonspecific binding. The target (IL-2) is added after washing. After capturing of the target molecules, a biotin-conjugated detection antibody is added to bind with the captured target molecules. Streptavidin-alkaline phosphatase is added to form a strong non-covalent interaction with the biotin via biotin bonding with streptavidin. Finally, ELF 97 substrate is added into the well, and fine, bright, and stable green fluorescent precipitates/aggregates form in situ within one to three hours (FIG. 7). These results show that the aggregates do not move, and provides evidence that one fluorescent dot is provided by one enzyme. The number of fluorescent dots can therefore be correlated to the number of target molecules and the concentration based on the sample volume.

In another aspect, the biotin-streptavidin portions of the report complex can be eliminated. In this aspect, as shown in FIG. 7 (top panel) a reporter enzyme, for example alkaline phosphatase or other reporter enzyme, can be conjugated to the detection antibody via known methods.

In still another aspect, the capture antibody can be functionalized to provide a desired property. For example, as shown in FIG. 7 (top and bottom panels) the capture antibody can be linked to a magnetic bead. A magnet-assisted purification step can then be used at any point in the process, for example, to purify the immunocomplex-ALP from other components.

Use of the ultrasensitive detection method with a detection complex including fluorescent beads has been demonstrated, again using IgG, prostate PSA, and IL-2 as a model ELISA assay. In this detection method, the capture antibody (IgG) is first coated on a 96-well microplate, followed by BSA blocking. After removing the blocking agent, the sample containing target molecules (PSA) is added and the antigen is captured by the anchored capture antibody. To functionalize the fluorescent beads with detection antibody, carboxyl-group functionalized fluorescent microbeads (500 nm to 1 μm) having strong green fluorescence used. The carboxyl groups are activated by EDC to form an amine-reactive O-acylisourea intermediate, which is mixed with the IL-2 detection antibodies to covalently conjugate the microbeads and the detection antibody. A glycine solution is used to quench the unreacted active sites on the microbeads after the antibody conjugation. Finally, the as-prepared detection antibody-conjugated microbeads are added to the microwells to form a sandwich immunocomplex as shown in FIG. 8. After removing the nonspecific bound fluorescent microbeads by extensive washing, direct counting of the fluorescent microbeads is conducted using a scanning microscope (FIG. 8, bottom panel) (here, a Nikon confocal microscope). Each fluorescent dot represents a single immunocomplex, corresponding to one captured target biomarker.

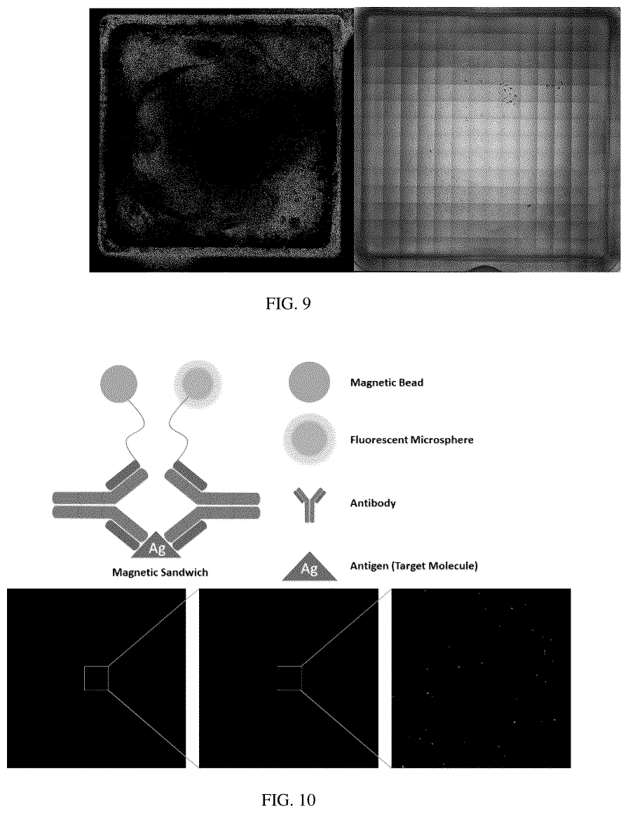

As stated above, the scanning microscope images can further be analyzed to determine numbers and concentration of the target biomolecule. With reference to FIG. 9 (bottom panel), scanning and counting of the fluorescent beads is conducted. The number counted is 6.44×10⁵, which is in good agreement with the number of PSA molecules loaded (calculated total number loaded is 7×10⁵).

Use of the ultrasensitive detection method with a magnetic microbead-derivatized capture antibody and a detection complex including fluorescent beads has been demonstrated using IgG, prostate PSA, and IL-2 as a model ELISA assay (FIG. 10). In this method, amine groups are pre-coated on the surface of both beads, which allows proteins (e.g., antibodies) containing carboxylate groups to be conjugated to the bead. Proteins are dissolved in 1-ethyl-3-[3-dimethylaminopropyl] carbodiimide (EDC) in a 2-(N-morpholino)ethanesulfonic acid (MES) buffer solution to activate the carboxyl group of the protein. After the activation, the beads are mixed with the respective activated proteins. As the conjugation completed, glycine solution is added to quench the unreacted active sites. One or more rounds of centrifugation can be used to purify the detection antibody fluorescent microbead conjugates, while one or magnetic-assisted washing can be used to purify the capture antibody magnetic bead conjugates. After the separation, two the kinds of as-prepared beads and the target molecules are mixed, for example in one tube to form the sandwich immunocomplexes. After magnetic-assisted separation of immunocomplexes and excess magnetic beads from excess fluorescent beads, a direct count can be carried out to identify the number of fluorescent immunocomplexes.

In another aspect of the method to separate excess fluorescent beads after formation of the immunocomplexes, the solution containing the immunocomplexes can be placed on an upper surface of a density gradient medium having a specific density gradient greater than fluorescent beads and water (e.g., OptiPrep™, to form cocktail ‘cocktail’ configuration in the 96 microplate. A magnet block located at the bottom of the target well of the microplate attracts the immunocomplexes and unbound magnetic beads into and through the density media towards the magnet, while the unbound fluorescent microbeads remain in the top layer. The single molecule immunocomplex can then be readily characterized using an optic fluorescent microscope.

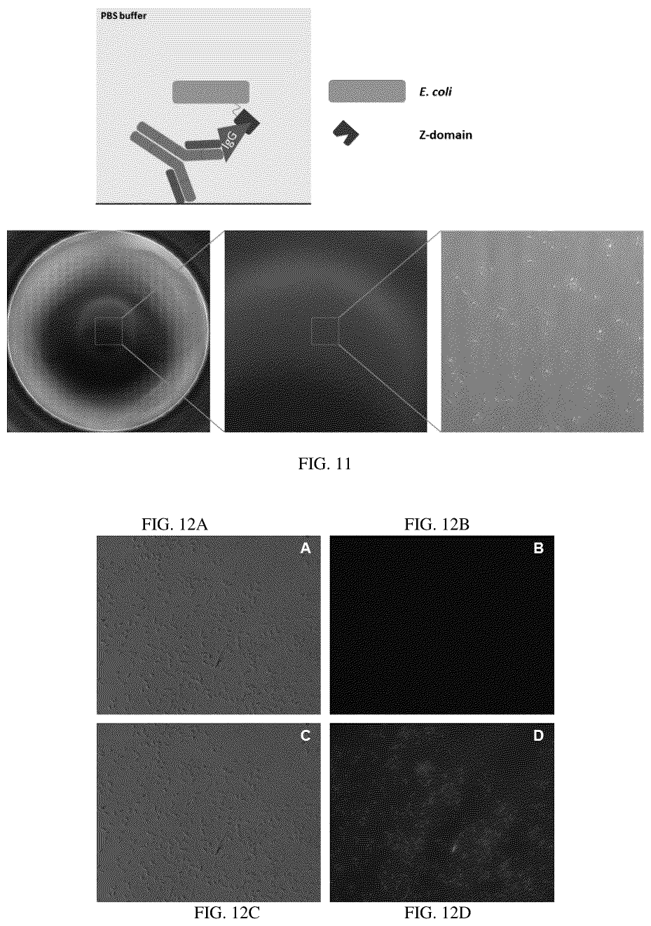

Use of a bacterium with a surface-expressed antibody binding domain as the reporter complex has also been demonstrated (FIG. 11, top panel). After immunoreaction and the binding of E. coli, the number of bacteria can be directly counted using scanning microscope and correlated to the concentration of target molecules. In particular, a model system used a scanning-based IgG detection method based on genetically engineered E. coli containing an antibody-binding element (a Z-domain) as the reporter. IgG is used as both antigen and antibody for Z-domain interaction. The Z domain is derived from the B-domain of protein A, which has been shown to bind IgG with a high affinity. Thus, the E.coli that contains a Z-domain on the outer membrane can be an excellent reporter due to its large size compared to other molecular reporters. The method is easy to perform because it is similar to conventional ELISA. The anti-IgG capture antibody is incubated within the 96 microplate to establish the capturing surface. BSA is added to block the non-specific binding sites. After blocking, the target molecules, IgG, are loaded into the well to bind with the capture antibodies. Finally, E. coli with surface-expressed Z-domains are added to form immunocomplexes. After washing any excessive E. coli, the entire well is subject to counting to obtain the number of the target molecules. This result is convenient to convert to the accurate concentration of IgG.

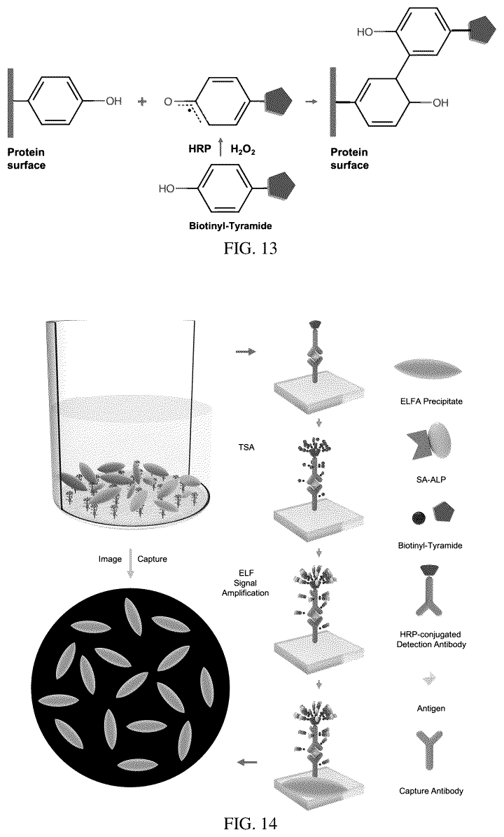

A major advantage of this method is that bacterial reporters can be used as a universal signal amplifier and easily prepared through facile cell culturing with low cost. Moreover, in an aspect, the bacteria can also be either stained to have fluorescence (using, e.g., Nile Red, FIG. 12) or genetically engineered to self-express fluorescent reporters (e.g., fluorescent proteins) for easier or more accurate resolution and counting results. In still another aspect of the ultrasensitive method for detecting biomolecules, an additional amplification method can be used in combination with the above-described signal amplification to provide a dual amplification counting (DAC) ELISA. In particular, tyramide signal amplification (TSA) can be used in combination with the formation of fluorescent aggregates as described above, followed by imaging. TSA has been described for signal amplification, and is advantageously a fast turnaround, high reliability, and convenient technique that has been used with in-situ hybridization, electron microscopic immunocytochemistry, cell labeling, tissue signal amplification, and like, for low-abundance biomolecules of interest, but has heretofore not been compatible with conventional plate-based ELISA platforms in general. However, as described herein, TSA can be readily used in combination with the aggregate-forming and scanning method described above. FIG. 13 is a schematic showing the TSA reaction.

The DAC-ELISA method includes two amplification steps, the first being TSA and the second being the ALP-enabled ELF signal amplification. As illustrated schematically in FIG. 14, a capture antibody is immobilized on a fixed surface, e.g., a plate or a well of a microplate, exposed to an antigen from a sample to be tested, and then exposed to a horseradish peroxidase (HRP)-conjugated detection antibody. This construct is then exposed to biotinyl-tyramide (B-T) to provide conjugation to the detection antibody. In the presence of H₂O₂, B-T is oxidized by HRP to form tyramide radicals. These radicals further react with electron-enriched molecules such as tryptophan and tyrosine on biomolecules (e.g., proteins, antibodies, and the like) in the proximity of biotin. As this reaction has no selectivity, it labels the detection antibody, antigens, and capture antibody in proximity with biotin. The DAC-ELISA method allows detection of a single molecule. A lower LOD of DAC-ELISA can be 50 picogram per milliliter (pg/mL), or 100 pg/mL, or 500 pg/ml, or 1000 pg/mL.

To further amplify and generate the signal, ALP-enabled ELFA signal amplification is introduced after TSA as described above. As shown in FIG. 13, the more biotins that are conjugated onto the immunocomplex, the more reporter enzymes are anchored through the interaction between biotin and SA-ALP. Upon the addition of the substrate (ELFA-saturated ELFP solution), ALP converts ELFP to ELFA and to form fluorescent precipitates with superior photostability that can attach to the bottom surface of the surface (e.g., a plate or microwell). The precipitates can be imaged, and their total number can be counted and correlated with the concentration of analytes. In clinical practice, analyte standards can be used to establish the calibration curve, and thus the concentrations in unknown samples can be obtained accordingly. In this method, the signal is amplified by TSA and ELF amplification in tandem, which significantly lowers the limit of detection of the method, comparing to the conventional ELISA.

The methods has been demonstrated by ultrasensitive detection of mouse total IgG (a model biomarker). Conventional sandwich immunocomplex constructed in the microplate wells was used as a base. The dual amplification strategy was accomplished through tyramide signal amplification and ALP-enabled formation of fluorescent precipitates. The first amplification relies on biotinyl-tyramide (B-T) conjugating with the sandwich immunocomplex (triggered by HRP) to increase the number of biotins, which can further increase the number of reporter enzymes (ALP) through the interaction between biotin and streptavidin-ALP (SA-ALP), thus resulting in the second amplification through rapid fluorescent precipitates formation upon the addition of ELFA-saturated ELFP substrate. The rapid counting of fluorescent precipitates in one or more wells, for example 1 to 100, or 2 to 80, or 3 to 70, or 4 to 50, or 5 to 40, or 10 to 35, for example 25 images of each well can be correlated to the concentration of IgG with an excellent limit of detection (LOD) compared to that of conventional ELISA. Mouse IgG spiked in goat serum samples was investigated to further evaluate its specificity, recovery, accuracy, and precision.

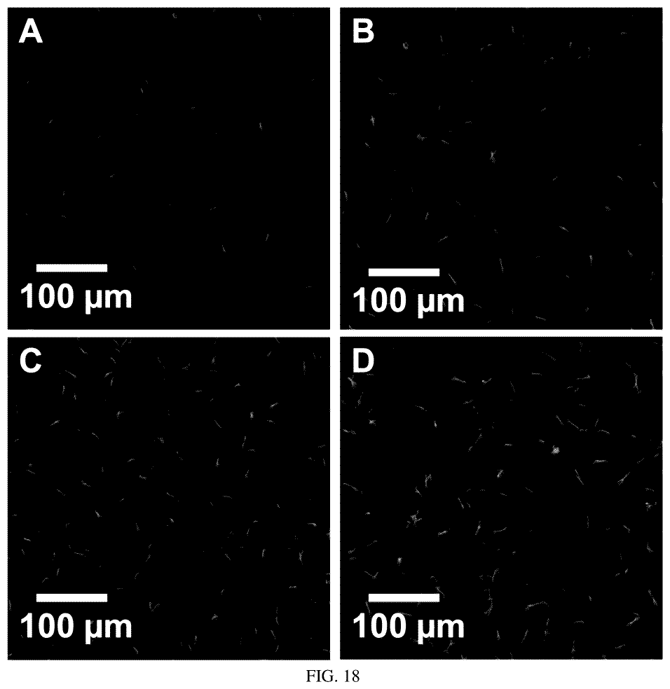

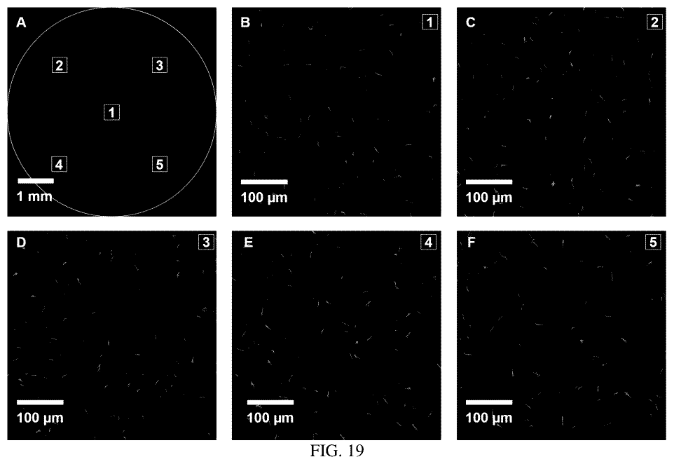

TSA significantly enriches the number of ALP on one antigen-antibody binding event, while ALP-enabled fluorescent precipitate formation allows easier direct-counting of the precipitates for concentration correlation of target molecules. Thus, where one-step ALP single amplification does not return sufficient signal (e.g., fluorescent precipitates), TSA can be used to further amplify the signal. To shorten the assay time and enhance the sensitivity, ELFA-saturated ELFP substrate solution is used in conjunction with the optimal substrate concentration and incubation time. Under the optimal conditions, detection of mouse IgG with high sensitivity, accuracy, and precision was obtained. Current protocol is adequate for real applications, corroborated by the good recovery test result using spiked samples. However, it is noted that even when only 25 images were captured for each well, the number of ELFA precipitates in these 25 images can be used to represent the signal for concentration correlation. The main rationale relies on the relatively uniform distribution of ELFA precipitates across the well, indicating there is no need to conduct time-consuming whole-well scanning. As shown in FIG. 18, 25 images at five different spots in a well were captured (FIG. 19 at A). Counting results of these five spots were used to conduct the single-factor ANOVA test. The p-value was 0.07, indicating no significant difference among the different spots tested. The other rationale of only capturing 25 images (instead of the whole well) is that a shorter time can be used for image capturing. For example, it can take about 50 seconds to capture 25 images, while 7 minutes may be needed to scan each whole well.

In addition, where triplicate experiments, for example on multiple IgG concentrations in each round are used, the hours to complete the scanning can cause a significant difference in incubation time at different wells for imaging and determining the corresponding precipitate number, since the enzymatic reaction is a continuous process. This delay can negatively affect assay accuracy. Accordingly, in an embodiment, a quenching agent is added to terminate the enzymatic reaction. Quenching agents known for use in conventional ELISA can be used. For example, ethylenediaminetetraacetic acid (EDTA) and L-phenylalanine are two inhibitors that are widely used with ALP. The addition of these inhibitor solutions can quench the enzymatic reaction, thus allowing direct-counting of the whole well without the aforementioned delay. However, as shown in the Examples below, the distribution of the ELFA precipitates can be extensively disturbed by the addition of bulk quenching solution, resulting in inaccuracy in counting. Thus, more time may be needed for the disturbed aggregates to redisperse, resulting in a longer assay time. Therefore, the accuracy achieved by use of a quenching agent is used to terminate the enzymatic reaction, it should be balanced against the increased time for settling. When used, care should be taken to minimized or avoid disturbing the precipitates.

In another aspect, to further shorten the turnaround time of the entire assay, strategies are not limited to the optimization of the assay steps described herein. All other ELISA optimization strategies such as varying the capture/detection antibody concentrations, finding the shortest blocking time, and the like can be applied to the single or dual amplification method.

A system for performing the ultrasensitive method includes an optically transparent surface for support of the amplified signal; and a counting detection device for counting the aggregates. In an aspect, the system includes an optically transparent surface for supporting ELFA precipitates, the SA-ALP as the reporter enzyme, and a counting detection device such as a fluorescent microscope with scanning function and an appropriate filter. In another aspect, the system includes an optically transparent surface for supporting ELFA precipitates from DAC-ELISA, an HRP-labeled detection antibody as a catalyst for TSA, B-T, the SA-ALP as the reporter enzyme, and a counting detection device such as a fluorescent microscope with scanning function and an appropriate filter. All these instruments/reagents are commercially available and generally available or accessible in analytical laboratories. The concentrations of B-T, SA-ALP, and ELFP substrates can be adjusted according to the need for detection sensitivity and the assay time. Another advantage of the DAC-ELISA system is the convenience as all reagents being commercially available with quality control. Thus it does not require additional complicated synthesis as in other nanoparticles-based strategies.

DAC-ELISA can also be integrated with bead-based ELISA to provide a new digital detection technique with ultra-sensitivity, using the magnetic beads as described above. When the bead concentration is low, the TSA amplification only occurs on a bead bearing an individual immunocomplex without cross-reaction reaction onto other beads, due to the short lifetime of tyramide radicals. In addition, ELFA precipitates tend to localize at the enzymatic activity sites rather than diffusing to other locations

In summary, ELISA is a predominant technique in the detection of biomarkers. Notwithstanding its ubiquity and numerous advantages, its application for detection of low-abundance biomarkers requires ultrasensitivity. Described herein is an amplification and dual amplification strategy that provides imageable results for ready and rapid quantitation that is compatible with conventional plate-based ELISA. Advantageously, the total material cost of this method can be low. For example, 0.5 mg antibody can be used to complete at least 200 independent experiments. All other materials, such as the glass chamber, chemicals, and other consumables are all commercially available, inexpensive, and easy to obtain. The method is easy and does not require skilled personnel. There is no complicated procedure involved in the detection and scanning. All the counting process can be achieved automatically (digitally) by a scanning microscope. This method can be applied for any biomolecules of interest given that there is an existing antibody or DNA/RNA probe or ligand for the target biomolecules.

DAC-ELISA was further developed and validated for ultrasensitive detection of biomarkers using mouse total IgG as a model compound. Due to its compatibility with conventional plate-based ELISA, the dual amplification strategy opens a new and universal avenue to improve the sensitivity of conventional plate-based ELISA and holds great potential in achieving ultrasensitive detection of biomarkers on conventional plate-based ELISA platform. The dual amplification strategy is accomplished through the combination of tyramide signal amplification and alkaline phosphatase-enabled formation of fluorescent precipitates, while the rapid counting of fluorescent precipitates can correlate the number to the concentration of IgG with a good linearity. Under the optimal conditions, the limit of detection of the commercial mouse IgG kit (1.56 ng/mL) can be extended to an LOD of 54.5 pg/mL. Recovery tests demonstrate the reliability of the method. DAC-ELISA can be employed where use of additional signal amplification strategies such as TSA or nanoparticles alone in plate-based ELISA does not overcome resolution limitations. Another advantage of DAC-ELISA is that there is no compartmentalization required compared with the existing digital ELISA. Also, current bead-based digital ELISA requires a high binding affinity between antibody-antigen pairs with a K_{D} value usually below 10⁻¹² to survive through rigorous washing steps. However, in the present study, IgG antibody typically possesses a K_{D} value in the nM range and the results indicate that the developed dual amplification enabled counting based system may be applicable for antibody-antigen pairs with low affinity.

The methods are further illustrated by the following Examples, which are not intended to limit the claims.

### EXAMPLES

### Reagents and Instruments

IgG (Total) Mouse Uncoated ELISA Kit, Streptavidin-Alkaline phosphatase conjugate (SA-ALP), and ELF™ 97 phosphate (ELFP) were from Thermo Fisher Scientific Inc. (USA). ELAST ELISA Amplification Kits were from PerkinElmer Life Sciences, Inc. (USA). Bovine serum albumin (BSA) was from Sigma-Aldrich (USA). 10× phosphate buffered saline (PBS, pH=7.4) and 10× Tris/Glycine/SDS buffer (pH=8.3) were from Bio-Rad Laboratories, Inc. (USA). 10× buffers were diluted to 1× before use. H₃PO₄, Goat Serum, Tween™ 20, Syringe filters (pore size, 0.2 μm), and Falcon™ 96-well (flat bottom) polystyrene microplates were from Fisher Scientific Company (USA).

Colorimetric readouts were acquired using Synergy™ HT Multi-Detection Microplate Reader (Bio-Tek Instruments).

Images were captured using the BZ-X800 All-in-One fluorescence microscope (Keyence Corporation) with a 20× objective lens (NA=0.45, Nikon). A customized optical filter cube with λₑₓ=345 nm and λₑₘ=530 nm was obtained from Chroma Technology Corporation. All images were processed using the BZ-X800 analyzer software.

### Protocol for DAC-ELISA

The procedure for DAC-ELISA includes four steps in sequence, including a sandwich ELISA step, a TSA amplification step, an ALP-enabled ELF amplification step, and image capture for direct-counting.

Sandwich ELISA Step. The protocol used in this step was adapted from the manual of the purchased ELISA kit. In brief, 1000 μL capture antibody solution was added to each well of the 96-well microplate of the kit. The plate was sealed and incubated overnight at 4° C. After overnight incubation, solutions were aspirated, and the capture antibody-coated wells were washed twice using 300 μL wash buffer (1×PBS, 0.05% Tween™ 20). After washing, all wells were blocked with 2500 μL blocking buffer provided in the ELISA kit for 2 hour (hr) at room temperature. The blocking solution was then aspirated, and all wells were washed twice using the wash buffer. Next, 1000 μL of the analyte dissolved in assay buffer (a component of the ELISA kit) with varying concentrations was added into the well, denoted as experimental wells, while 1000 μL assay buffer was loaded into the control wells. After the addition of analyte dissolved in assay buffer and blank assay buffer, 50 μL HRP-conjugated detection antibody solution was added into each well. The plate was incubated for 2 hr at room temperature. Then, the solutions were aspirated, and wells were washed with wash buffer for four times.

TSA Step. The protocol used in this step was adapted from the manual of the amplification kit. Wells prepared in the sandwich ELISA were incubated with biotinyl-tyramide (B-T) solution (5 μL/mL) in the presence of H₂O₂ for 15 minutes at room temperature. After aspiration, all wells were washed with 1×PBS, 0.5% Tween™ 20 for four times.

ALP-enabled ELF amplification. Wells prepared in sandwich ELISA followed by TSA were then incubated with 100 μL of 2 μg/mL SA-ALP dissolved in PBS with 1% BSA for 15 minutes at room temperature. Solutions were aspirated, and all wells were washed using 1×PBS, 0.5% Tween™ 20 for four times. After washing, 100 μL of 16.67 μM ELFP Tris/Glycine/SDS solution (ELFA saturated) was added into each well. The plate was incubated for 2.5 hr at room temperature before image capture.

Image capture for direct-counting. The microplate was fixed on the stage of the BZ-X800 microscope for imaging. For each well or area of interest, 25 images were captured, and the number of ELFA precipitates in each image was counted employing BZ-X800 analyzer software.

### Example 1

Optimization of B-T concentration for TSA. Wells were first prepared using the sandwich ELISA protocol, and the analyte dissolved in assay buffer were 2-fold serial diluted mouse IgG standard (a component of the ELISA kit) from 1.5625 ng/mL to 0.0977 ng/mL. Wells prepared in sandwich ELISA were then incubated with 50× (20 μL B-T solution/mL), 100× (10 μL/mL), 200× (5 μL/mL) diluted B-T solutions in the kit for 15 minutes at room temperature. After aspiration, all wells were washed with 1×PBS, 0.5% Tween™ 20 for four times. After washing, each well was incubated with 100 μL SA-HRP (a component of the ELISA amplification kit) for 15 minutes at room temperature. All wells were then aspirated and washed using 1×PBS, 0.5% Tween™ 20 for four times. Finally, wells were incubated with 1000 μL TMB substrate solution for 15 minutes at room temperature. 1000 μL of 1 M H₃PO₄ (aq) was finally added to quench the reaction. The plate was read at 450 nm employing a microplate reader. Experiments were conducted in triplicate. The optimal B-T concentration (5 μL/mL) which was experimentally determined to generate the best amplification was employed in TSA in this study.

### Example 2

Optimization of the Substrate. Preparation of ELFA-Saturated ELFP solution. 100 μL of 100 μM ELFP Tris/Glycine/SDS solution was mixed with 100 μL of 2 μg/mL SA-ALP dissolved in 1×PBS with 1% BSA for 15 minutes. Then, the mixture was incubated in a 100° C. water bath for 1 hr to inactivate the ALP. The temperature was reduced to room temperature before further use. ALP inactivated mixture was vortexed for 30 s. 100 μL mixture was then added to 4,900 μL ELFP Tris/Glycine/SDS solution where the final concentration of ELFP is 16.67 μM. This solution was incubated for 10 minutes and then filtered with a 0.2 μm syringe filter to separate un-dissolved ELFA precipitates. The filtrate obtained was an ELFA-saturated ELFP solution, which was ready for use.

### Example 3

The effect of ELFA saturation in ELFP solution on fluorescent precipitates formation. Two wells were prepared using the DAC-ELISA protocol. The analyte dissolved in assay buffer was 100 μL of 1.5625 ng/mL mouse IgG standard (a component of the ELISA kit). Modifications were in the ELF amplification step where one well was incubated with 100 μL of 16.67 μM ELFP Tris/Glycine/SDS solution (ELFA saturated), and the other was incubated with 100 μL of 16.67 μM ELFP Tris/Glycine/SDS solution. Images were captured every 30 min during 3 hr incubation. Experiments were conducted in triplicate.

### Example 4

Determination of optimal ELFP concentration. Wells were prepared employing the DAC-ELISA protocol. The test was based on a 100 μL assay buffer for background signal as minimal background signal is critical for improving the limit of detection. Modifications were in the ELF amplification step where wells were incubated with 100 μL of 50 μM, 25 μM, 16.67 μM, and12.5 μM ELFP Tris/Glycine/SDS solution (ELFA saturated) respectively. Experiments were conducted in triplicate.

### Example 5

Determination of optimal incubation time. Wells were prepared using the DAC-ELISA protocol. The analyte dissolved in assay buffer was 100 μL of 1.5625 ng/mL mouse IgG standard in the ELISA kit. Modifications appeared in the image capture step, where images were captured every 30 min during 4 hr of incubation. Experiments were conducted in triplicate.

### Example 6

Matrix effect and recovery test. For the study of the matrix effect, wells were prepared employing the DAC-ELISA protocol. The analyte was 100 μL diluted goat serum in assay buffer. Goat serum was diluted 4, 10, 100, 500, 1,000, 10,000, and 100,000 folds using the assay buffer. In the recovery test, wells were prepared employing the DAC-ELISA protocol. For the study of the recovery test, the analyte was 100 μL of IgG standard spiked into 100,000 times diluted goat serum in assay buffer where the final concentrations of IgG standard are 0.1 ng/mL and 0.2 ng/mL, respectively.

### Results and Discussion, Examples 1-6

Comparison of the effect of ELFP substrate and ELFA-saturated ELFP substrate. FIG. 14 shows a comparison of the effect of ELFP substrate and ELFA-saturated ELFP substrate on fluorescent precipitates formation without (top panels) and with (bottom panels ELFA-saturated ELFP substrate. Using an ELFA-saturated ELFP substrate is advantageous in the DAC method, which shortens the assay time and enhances the sensitivity. Usually, ELFA is produced as ALP catalyzes ELFP substrate. Although ELFA is water-insoluble, it does not precipitate out from the solution until the solution is saturated with ELFA. If only ELFP solution is used, a different time is required to generate ELFA-saturated solution, because the number of ALPs is dependent on the analyte concentration. Since ELFA precipitation starts at different moments, the correlation between ALP and ELFA precipitate is not as reliably established among different target concentrations. The use of ELFA-saturated ELFP avoids such differences. Once the ELFA-saturated ELFP is added, the ELFA precipitate will separate from the solvent immediately regardless of the amount of ALP. In addition, the utilization of the ELFA-saturated ELFP substrate can shorten the assay time by eliminating the saturation step, which can be especially useful for the detection of the low abundance target. As illustrated in FIG. 14, the number of ELFA precipitates produced from the use of the ELFA-saturated ELFP substrate is more than that of the unsaturated one under the same incubation time. Therefore, an ELFA-saturated ELFP substrate is used in subsequent experiments.

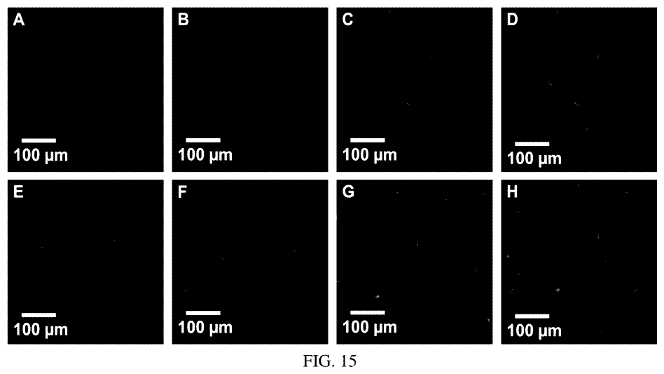

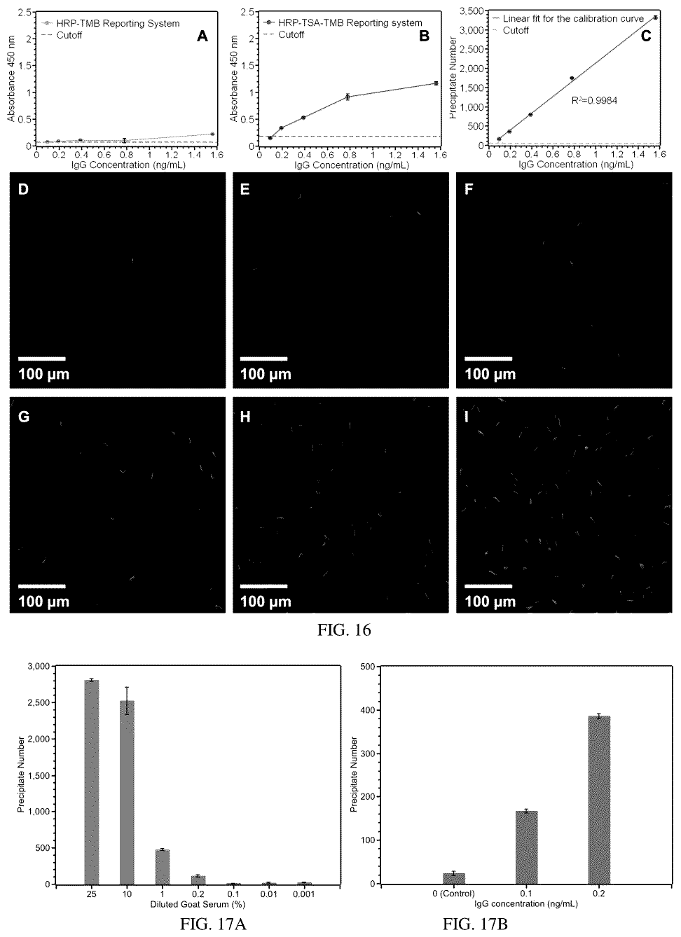

Performance of DAC ELISA in Mouse IgG Standard Test. The main goal of this study is to develop the DAC-ELISA system to improve the LOD of the conventional plate-based ELISA with a minor modification of the protocol. The LOD of the conventional ELISA kit employed in this study is 1.56 ng/mL. The IgG standards tested in the current study were diluted from 1.56 ng/mL in a 2-fold serial manner, and the wells with blank assay buffer were prepared as negative controls. Parallel tests were conducted employing the original HRP-TMB reporter system, TSA-HRP-TMB reporter system, and DAC reporter system. For the original HRP-TMB reporter system described in the kit (FIG. 15, top panel at A), all absorbance values obtained at 450 nm were below 0.25. These readouts were so small and close to the cutoff value (0.065±0.002) and the resolution of the microplate reader, indicating that the detection of IgG lower than 1.56 ng/mL is not achievable using original HRP-TMB system. This observation is in good agreement with the LOD of the kit. For the HRP-TSA-TMB reporter system (FIG. 15, top panel at B), signals were amplified to a certain extent compared to the HRP-TMB system, which is attributed to TSA. However, the cutoff value (0.183±0.004) increased and was larger than the readout of 0.148±0.019, corresponding to 0.098 ng/mL IgG standard. In addition, no strong linear relationship was observed between the absorbance and the tested concentrations range. These results indicate that these two reporter systems may not be reliable to be applied for biomarker detection at such low concentrations. As shown in FIG. 15, top panel at C, the DAC reporter system surpassed the other two strategies and lowered the LOD of the conventional ELISA over 25 times. In addition, a good linear fit was achieved for the IgG standards with the concentration ranging from 98 pg/mL to 1.5625 ng/mL. The LOD was 54.5 pg/mL, calculated by extrapolating the concentration from the readout equal to background readout plus three standard deviations of the background readout. The coefficient variations (CV) of all values were ranging from 1.25% to 2.41%, which represented the superior precision and repeatability of the results. Moreover, the large slope of the calibration curve between the precipitate number and the analyte concentration indicates enhanced sensitivity and accuracy since the readout number varies substantially even though the concentration of analyte just has a minor variation FIG. 15, middle and bottom panels at D-I).

Matrix effect, Accuracy, and Precision. Elimination of matrix effect is desirable for specimens from body fluids such as plasma and serum, due to the fact that the complicated components in biological samples might result in high background noise and thus cause overestimated results. Currently, the predominant strategy to eliminate the matrix effect is through direct dilution of the sample into assay buffer. In the commercial mouse IgG ELISA kit, the supplier recommends the starting dilution is 10,000-fold for serum and plasma samples using assay buffer. To evaluate the matrix effect in the current study, goat serum (free of mouse IgG) was used and diluted in assay buffer provided in the kit from 4- to 100,000-fold. As illustrated in FIG. 16A, the precipitate numbers were decreased with the increase of dilution factors, as expected. The average precipitate number with dilution factors of 1,000, 10,000, and 100,000 was counted and calculated to be as low as 10, 19, and 23, respectively. This result indicates that the matrix effect of biological samples could be ignored by a substantial dilution. Thus, 100,000-fold diluted goat serum with spiked mouse IgG was used in subsequent recovery tests.

To carry out the recovery tests, the DAC-ELISA method as described herein was challenged with 100,000-fold diluted goat serum spiked with 0.1 ng/mL and 0.2 ng/mL mouse IgG. The results of the IgG standard spiked goat serum test (n=3) show superior accuracy, precision, and recovery (FIG. 16B). For the 0.2 ng/mL and 0.1 ng/mL samples tested, the recovery reached 99.33 ±2.43% and 97.90 ±4.01%, respectively. The small relative error was 0.67% and 2.10%, and the small relative standard deviation was 2.45% and 4.10%, respectively (Table 1). These results indicated the reliability and excellent performance of the DAC-ELISA method. The Table shows the performance of DAC-ELISA in the recovery tests, where mouse IgG standards were spiked into 100,000-fold diluted goat serum (n =3).

| | Reference | Detected | Relative | | |
|---|---|---|---|---|---|
| Target | concentration | Concentration | Error | Reproducibility | Recovery |
| Molecule | (ng/mL) | (ng/mL) | (RE %) | (RSD %) | (%) |
| Mouse IgG | 0.2 | 0.1987 ± 0.0049 | 0.67 | 2.45 | 99.33 ± 2.43 |
| Mouse IgG | 0.1 | 0.0979 ± 0.0040 | 2.10 | 4.10 | 97.90 ± 4.01 |

DAC-ELISA Optimization in Assay Development. The above experiments were conducted under optimized conditions. The selection of optimal substrate concentration and the determination of the substrate incubation time are presented below. Identifying an appropriate substrate concentration is important because it determines the background signal (noise) level for the negative control, which can substantially influence the sensitivity of the assay, which is a trade-off between the substrate concentration and the precipitate formation (rapidness and countability). On the one hand, a higher substrate concentration results in large numbers of ELFA precipitates generated in a short time. However, more precipitates (noise) for negative control decrease the signal-to-noise ratio and consequently decrease the sensitivity of the assay. On the other hand, a low substrate concentration requires an extended assay time to form precipitates.

Accordingly, four substrate concentrations ranging from 12.5 μM to 50 μM were tested with an incubation time of 2.5 hours. As illustrated in FIG. 17, the numbers of ELFA precipitates decreased with the decrease of concentrations of ELFP. At the concentration of 50 μM (Figure S3A), a higher number of precipitates were produced but intertwined with each other, which causes difficulty in counting accuracy. Once the concentration was equal to or lower than 16.67 μM (Figure S3C and D), it is observed that nearly no ELFA precipitate appeared, which was an ideal situation for the negative control. Therefore, 16.67 μM ELFP solution (ELFA saturated) was used in the assay.

Conducting the assay with a proper substrate incubation time can be significant as well. Being similar to the effect of substrate concentration on the performance, it is also a trade-off between substrate incubation time and the precipitate formation (rapidness and countability). On one hand, a short substrate incubation time may result in insufficient ELFA precipitates produced, which causes the failure of the assay. On the other hand, excessive ELFA precipitates associated with extended incubation time intertwine with each other and then form aggregates, resulting in counting inaccuracy and underestimating the precipitate number. In addition, a long incubation time is not desired since it results in a long assay time.

To determine a suitable substrate incubation time, a time lapsing study lasting 4 hours was conducted to monitor the precipitate layout on the well bottom. As illustrated in FIG. 18, ELFA precipitates began to appear after 1 hr incubation, and its number increased gradually with the incubation time to 2 hr. However, 3 hr incubation results in abundant ELFA precipitates with a few precipitates intertwined, and the intertwinement becomes more significant at 4 hr incubation (FIG. 5C-D). To maximize the number of ELFA precipitates for a better sensitivity while minimizing the intertwinement of precipitates for better counting accuracy, 2.5 hr incubation time was used in the assay development.

Sampling rather than whole-substrate counting can also be optimized. In well as shown at FIG. 18A, 25 images at five different spots (FIG. 18B, FIG. 18C, FIG. 18D, and FIG. 18E) were captured. Counting results of these five spots were used to conduct the single-factor ANOVA test. The p-value was 0.07, indicating no significant difference among the different spots tested. The other rationale of only capturing 25 images (instead of the whole well) is to consider the time required for image capturing.

The various aspects of the invention are illustrated by the following embodiments, which are not meant to limit the claims.

In an embodiment, a method for detecting a target molecule, preferably a biomolecule such as antigen, a peptide, a protein, an enzyme, a DNA, an RNA, a virus, or a bacteria, more preferably an antigen, in a sample comprises: providing a capture species immobilized on a fixed surface, preferably a capture antibody; exposing the immobilized capture species to the sample to bind the target molecule; exposing the bound target molecule to an amplifying reporter complex to bind the reporter complex to the bound target molecule; and imaging all, a portion, or a plurality of portions, of the fixed surface, preferably by scanning microscopy, to count the number of the reporter complexes or the number of an imageable product derived from each reporter complex. Preferably in this embodiment, each of the counted reporter complexes or product derived from each reporter complex corresponds to one of the bound target species. In a further aspect in this embodiment, the reporter complex comprises a detection antibody linked to an imageable microbead, for example a fluorescent or colored microbead, to an imageable nanobead, for example a fluorescent or colored microbead, to a bacterium, or to a reporter enzyme that converts a plurality of enzyme substrates to a plurality of reporter molecules that provide the imageable product. In any of the foregoing, it is also possible to have the capture species (e.g, a capture antibody), the reporter complex (which can include a detection antibody), or both further include a magnetic microbead, a magnetic nanobead, or both. The imaging, for example by scanning microscopy can further include quantitation of the reporter of the reporter complex or product of the reporter complex. The lower limit of detection in this embodiment can be, for example, 0.01 femtomole (fM), or 0.1 fM, or 1.0 fM.

In another embodiment, a method for detecting a target molecule, preferably a biomolecule such as antigen, a peptide, a protein, an enzyme, a DNA, an RNA, a virus, or a bacteria, more preferably an antigen, in a sample comprises: providing a capture species immobilized on a fixed surface, preferably a capture antibody; exposing the immobilized capture species to the sample to bind the target molecule; exposing the bound target molecule to an amplifying reporter complex to bind the reporter complex to the bound target molecule; exposing the reporter complex to a substrate to provide a product that aggregates and precipitates on the surface; and imaging all, a portion, or a plurality of portions, of the fixed surface to count the aggregates that precipitate on the fixed surface, preferably using scanning microscopy. Preferably in this embodiment, one aggregate corresponds to one of the bound target species. Optionally, the capture species (for example a capture antibody), the reporter complex (which can include a detection antibody), or both further include a magnetic microbead, a magnetic nanobead, or both, wherein in this aspect, the method further includes a purification step using the magnetic microbead, nanobead, or both. The aggregate can be fluorescent or colored, and is preferably fluorescent. Specifically in this embodiment, the reporter complex can include a capture antibody linked to an alkaline phosphatase. Optionally, the capture antibody can be linked (e.g., conjugated) to biotin, which can bind to a streptavidin-conjugated alkaline phosphatase. The imaging, for example by scanning microscopy can further include quantitation of the aggregates. The lower limit of detection in this embodiment can be, for example, 0.01 femtomole (fM), or 0.1 fM, or 1.0 fM.

In still another embodiment, a method for detecting a target molecule, preferably a biomolecule such as antigen, a peptide, a protein, an enzyme, a DNA, an RNA, a virus, or a bacteria, more preferably an antigen, in a sample comprises: providing a capture species immobilized on a fixed surface, preferably a capture antibody; exposing the immobilized capture species to the sample to bind the target molecule; exposing the bound target molecule to a signal amplifying complex to provide a bound signal amplifying complex; amplifying the incidence of the bound target molecule via the amplifying complex to provide an amplified signal; exposing the amplified signal to a reporter complex to bind the reporter complex to the amplified signal; exposing the bound reporter complex to a substrate to provide a product that aggregates and precipitates on the surface; and imaging all, a portion, or a plurality of portions, of the fixed surface to count the aggregates that precipitate on the fixed surface, preferably using scanning microscopy. Preferably in this embodiment, one aggregate corresponds to one of the bound target species. Optionally, the capture species (for example a capture antibody), the reporter complex (which can include a detection antibody), or both further include a magnetic microbead, a magnetic nanobead, or both, wherein in this aspect, the method further includes a purification step using the magnetic microbead, nanobead, or both. The aggregate can be fluorescent or colored, and is preferably fluorescent. Specifically in this embodiment, the amplifying complex includes a detection antibody linked to horseradish peroxidase, and amplifying the signal comprises exposing the bound amplifying complex to peroxide and biotinyl-tyramide to provide the amplified signal, i.e., a plurality of biotin molecules bound to the target; exposing the amplified signal, e.g., the plurality of biotin molecules to a detection complex such as a streptavidin-conjugated alkaline phosphatase to bind the detection complex; exposing the detection complex to a substrate, for example ELFP. This embodiment can have a lower limit of detection of 1,000 pg/mL, or 500 pg/mL, or 100 pg/mL, or 50 pg/mL.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

When an element such as a layer, film, region, or substrate is referred to as being “on” another element, it can be directly on the other element or intervening elements may also be present. In contrast, when an element is referred to as being “directly on” another element, there are no intervening elements present.

In general, the compositions, methods, and articles can alternatively comprise, consist of, or consist essentially of, any ingredients, steps, or components herein disclosed. The compositions, methods, and articles can additionally, or alternatively, be formulated, conducted, or manufactured so as to be devoid, or substantially free, of any ingredients, steps, or components not necessary to the achievement of the function or objectives of the present claims.

Unless specified to the contrary herein, all test standards are the most recent standard in effect as of the filing date of this application, or, if priority is claimed, the filing date of the earliest priority application in which the test standard appears.

The endpoints of all ranges directed to the same component or property are inclusive of the endpoints, are independently combinable, and include all intermediate points and ranges. For example, ranges of “up to 25 wt %, or 5 to 20 wt %” is inclusive of the endpoints and all intermediate values of the ranges of “5 to 25 wt %,” such as 10 to 23 wt %, etc.

The term “combination” is inclusive of blends, mixtures, alloys, reaction products, and the like. Also, “at least one of” means that the list is inclusive of each element individually, as well as combinations of two or more elements of the list, and combinations of at least one element of the list with like elements not named.

Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A method for detecting a target molecule in a sample, the method comprising:
providing a capture species immobilized on a fixed surface;
exposing the immobilized capture species to the sample to bind the target molecule;
exposing the bound target molecule to an amplifying reporter complex to bind the reporter complex to the bound target molecule; and
imaging the fixed surface to count the number of the reporter complexes or the number of an imageable product derived from each reporter complex.

2. The method of claim 1, wherein each of the counted reporter complexes or product derived from each reporter complex corresponds to one of the bound target species.

3. The method of claim 1, wherein the target comprises a peptide, a protein, an enzyme, a DNA, an RNA, a virus, or a bacteria.

4. The method of claim 1, wherein
the capture species is a capture antibody,
the target molecule is an antigen, and
the reporter complex comprises a detection antibody linked to an imageable microbead, to an imageable nanobead, to a bacterium, or to a reporter enzyme that converts a plurality of enzyme substrates to a plurality of reporter molecules that provide the imageable product.

5. The method of claim 4, wherein the imageable microbead or the imageable nanobead is fluorescent or colored.

6. The method of claim 4, wherein the capture antibody, the detection antibody, or both further comprise a magnetic microbead, a magnetic nanobead, or both.

7. The method of claim 4, wherein the plurality of reporter molecules form the product as an aggregate that precipitates on the fixed surface.

8. The method of claim 7, wherein the aggregate is fluorescent.

9. The method of claim 4, wherein the reporter enzyme is alkaline phosphatase.

10. The method of claim 4, wherein the reporter complex comprises a detection antibody conjugated to biotin, and the reporter enzyme is a streptavidin-alkaline phosphatase.

11. The method of claim 10, wherein the substrate is 2-(5′-chloro-2-phosphoryloxyphenyl)-6-chloro-4(3H)-quinazolinone (ELFP).

12. The method of claim 1, wherein the imaging is by scanning microscopy.

13. The method of claim 1, wherein a portion or a plurality of portions of the surface is imaged.

14. The method of claim 1, wherein the imaging and counting is by scanning microscopy.

15. The method of claim 1, further comprising exposing the bound target molecule to a signal amplifying complex and amplifying the signal before exposure to the reporter complex.

16. The method of claim 15, wherein the signal amplifying complex comprises a detection antibody linked to horseradish peroxidase, and amplifying the signal comprises exposing the bound detection antibody-horseradish peroxidase to peroxide and biotinyl-tyramide to provide the amplified signal.

17. The method of claim 16, wherein the reporter complex comprises streptavidin-alkaline phosphatase.

18. The method of claim 17, wherein the bound streptavidin-alkaline phosphatase is exposed to 2-(5′-chloro-2-phosphoryloxyphenyl)-6-chloro-4(3H)-quinazolinone (ELFP).

19. The method of claim 15, having a lower limit of detection of 50 pg/mL.
